(19) United States
(12) Patent Application Publication
(10) Pub. No.: US 2024/0368595 A1
(43) Pub. Date: Nov. 7, 2024

(54) POLYMERIC NANOPARTICLE DRUG DELIVERY SYSTEM

(71) Applicant: BAYLOR UNIVERSITY, Waco, TX (US)

(72) Inventor: Panagiotis Koutakis, Waco, TX (US)

(73) Assignee: BAYLOR UNIVERSITY, Waco, TX (US)

(21) Appl. No.: 18/653,507

(22) Filed: May 2, 2024

Related U.S. Application Data
(60) Provisional application No. 63/464,056, filed on May 4, 2023.

(51) Int. Cl. C12N 15/113; A61K 9/51; A61P 9/00

(52) U.S. Cl. CPC C12N 15/113; A61K 9/5138; A61K 9/5153; A61K 9/5161; A61K 9/5192; A61P 9/00

(57) ABSTRACT

Polymeric nanoparticles encapsulating microRNA are prepared according to described methods. The nanoparticles serve as an improved drug delivery system for treatment of cardiovascular disease.

### REFERENCE TO A SEQUENCE LISTING

This application contains a Sequence Listing XML, which has been submitted electronically and is hereby incorporated by reference in its entirety. Said Sequence Listing XML, created on Jun. 11, 2024, is named Baylor 410 Sequence Listing.xml and is 6.4 KB in size.

### BACKGROUND

This application claims priority to U.S. Provisional Patent Application Ser. No. 63/464,056, entitled “Polymeric Nanoparticle Drug Delivery System,” filed May 4, 2023, the entire contents of which are hereby incorporated by reference.

The present disclosure relates to methods for drug delivery in treatment of patients with cardiovascular disease.

Peripheral artery disease (PAD) affects about 8.5 million individuals in the United States, primarily those over age 40, with an estimated 12-20% of the general population developing PAD by age 65. Moreover, the risk of cardiovascular mortality is increased in PAD patients with nearly 250,000 revascularization procedures and 100,000 amputations performed annually as treatment strategies. The annual financial burden of the disease per patient is about $12,000, which is significantly increased after an amputation procedure.

### SUMMARY

The present disclosure pertains to a drug delivery system useful in treating patients with cardiovascular disease.

In particular, the present disclosure pertains to a drug delivery system that efficiently delivers treatment in the form of small RNAs and improves the patient's recovery or represses disease progression. The delivery system described herein has the potential to slow down the disease process, reducing the need for revascularization operations or amputation. The methods and systems of the present disclosure can significantly reduce cost as an approved drug therapy.

MicroRNAs (miRs) are endogenous nucleotides noncoding RNA (˜21˜25 nucleotides), that can regulate posttranscriptional gene expression. miRs regulate gene expression by binding to the 3′ un-translated region of a target mRNA and thereby reducing mRNA expression or protein translation. Circulating miRs, serve as potential biomarkers for the diagnosis and prognosis of peripheral artery disease (PAD) and are potential target towards the development of individualized treatments. Several studies have focused on the role of miRs in coronary, carotid, and aortic disease, however only few studies have investigated the miRs as potential biomarkers in PAD patients. Circulating miR-210 is significantly elevated in PAD patients compared to healthy control subjects. Specifically, miR-210 is a target of Hypoxia-inducible factor 1-α, which directly activates its transcription under low oxygen tension. Activated miR-210 targets several transcripts involved in multiple aspects of cellular response to hypoxia, inhibiting apoptosis, repressing mitochondrial metabolism and promoting the shift from mitochondria respiration to glycolysis and inducing angiogenesis. Therefore, miR-210 is a master gene regulator under hypoxic conditions that can be used as a novel treatment for PAD.

### BRIEF DESCRIPTION OF DRAWINGS

FIG. 1 shows images of blood flow Doppler imaging at rest in mice injected with preferred embodiments of the polymeric nanoparticle drug delivery system, (A) before double ligation of the left femoral artery (HLI), (B) immediately after the HLI, (C) two weeks after HLI and (D) four weeks after HLI, where more shading means more blood flow.

FIG. 2 shows the net rate of mitochondrial oxygen consumption (JO2) (left panel) and H₂O₂ emission (JH2O2) (right panel) of the non-ischemic limb (NI as the absolute control; left), ischemic limb at 2 weeks post injection (Ischemia A; middle) and 4 weeks post injection (Ischemia B; right) for each of Complexes I (2,3), II, III, and IV.

FIG. 3 shows the effect of miR-210 mimetic (top row) and inhibitor (bottom row) at baseline, post ligation (HLI of femoral artery), three days (3-D), seven days (7-D), fourteen days (14-D), twenty-one days (21-D) and twenty-eight days (28-D) post ligation.

FIG. 4 shows miR-210 qPCR expression from ischemic (I) gastrocnemius, with data normalized to non-ischemic limb (NI).

### DETAILED DESCRIPTION OF PREFERRED EMBODIMENTS

The present disclosure relates to a drug delivery system and method for treatment of cardiovascular disease. In particular, the present disclosure relates to a polymeric nanoparticle drug delivery system.

In preferred embodiments, the drug delivery system described herein includes the delivery of microRNA. Preferably, miR-210 mimetic or inhibitor is used, but other suitable miRNAs may also be used, such as miR-1. The described methods utilize microRNA formed into nanoparticles which slowly release the mRNA into the area of interest. The nanoparticles can enclose any additional components besides the miRNA.

Preferred embodiments disclosed herein include methods for preparing embodiments of the polymeric nanoparticle drug delivery system. For example, preferred embodiments of preparing the polymeric nanoparticle drug delivery system can include preparing different solutions for mixture, such as a carrier solution, a nanoparticle-forming solution, and a miRNA solution. Preferred embodiments include a step of dissolving a carboxylic acid polymer such as polylactic-co-glycolic acid (PLGA) in a solvent such as chloroform or polyvinyl alcohol (PVA) to form a first polymer solution, such as a PLGA solution. Other carboxylic acid polymers in other solvents or stabilizers could be used. In additional steps a nanoparticle-forming biocompatible polymer such as chitosan, or other suitable nanoparticle-forming biocompatible polymer, is mixed with a solvent such as PVA or chloroform to form a second polymer solution, such as a chitosan solution. In preferred steps, undissolved material is filtered out of the solutions. Preferred embodiments of miRNA are miR-210, and preferably miR-210 mimetic or miR-210 inhibitor. The miRNA is preferably first mixed with the first or PLGA solution, then the resulting miRNA mixture is mixed with the second or chitosan solution to form a miRNA suspension. Additional steps include centrifuging the miRNA suspension and collecting the supernatant, which comprises the polymeric nanoparticles encapsulating miR-210. Additional stirring, sonication, centrifugation, and resuspension steps may be performed on the miRNA suspension prior to collecting the polymeric nanoparticles in a supernatant as preferred embodiments of the polymeric nanoparticle drug delivery system. The nanoparticles can be lyophilized and stored.

The lyophilized particles can be resuspended in a suitable carrier and administered to a patient by injection. The microRNA encapsulated in polymeric nanoparticles, prepared as described herein, is efficiently delivered to the patient for uptake. The microRNA miR-210 is strongly linked with the hypoxia pathway and transfection with miR-210 is known to promote positive effects in connection with cardiovascular disease and peripheral artery disease (PAD) in particular. The use of the polymeric nanoparticle system protects the miR-210 from degradation and provides a slow release of miR-210 over time.

Preferred embodiments of the polymeric nanoparticles used in the drug delivery system described herein include a polymeric sphere or capsule formed of, preferably, PLGA and chitosan with miR-210 encapsulated within the polymeric core. Additional preferred embodiments described herein include methods for treatment of cardiovascular disease, including peripheral artery disease (PAD), by administering a therapeutically effective amount of a composition comprising the polymeric nanoparticles encapsulating miR-210. The miR-210 can be miR-210 mimetic or miR-210 inhibitor. Results show that administration of preferred embodiments of the polymeric nanoparticles encapsulating miR-210 results in extended benefits, slow release of miR-210 and improved circulation following just one dose.

Additional preferred embodiments relate to a pharmaceutical composition for administration to a subject including a therapeutically effective amount of a composition comprising polymeric nanoparticles encapsulating miR-210 and a pharmaceutically acceptable excipient, adjuvant, carrier, buffer or stabilizer. A “therapeutically effective amount” means a nontoxic but sufficient amount of the drug to produce a positive biological effect on a cardiovascular disease being treated. The amount that is “effective” will vary from subject to subject, depending on the age and general condition of the individual, the particular concentration and composition being administered, and the like. Thus, it is not always possible to specify an exact effective amount. However, an appropriate effective amount in any individual case may be determined by one of ordinary skill in the art using routine experimentation. The actual amount, rate and time-course of administration will depend on the nature and severity of the disease being treated. Prescription of treatment is within the responsibility of general practitioners and other medical doctors. Furthermore, the effective amount is the concentration that is within a range sufficient to permit ready application of the formulation so as to deliver an amount of the drug that is within a therapeutically effective range. The pharmaceutically acceptable excipient, adjuvant, carrier, buffer or stabilizer should be non-toxic and should not interfere with the efficacy of the active ingredient. The precise nature of the carrier or other material will depend on the route of administration, which may be oral, or by injection, such as cutaneous, subcutaneous, or intravenous injection, or by dry powder inhaler. In preferred embodiments, the route of administration is by injection.

In pharmaceutical compositions for intravenous, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions using, for example, isotonic vehicles such as sodium chloride solution, Ringer's solution, or lactated Ringer's solution. Preservatives, stabilizers, buffers, antioxidants and/or other additives may be included as required.

In additional preferred embodiments, the pharmaceutical composition comprises appropriate dosages for the subject to whom the composition is administered.

### Example 1

An exemplary polymeric nanoparticle drug delivery system for delivery of miR-210 was prepared. In an initial preferred step, 95 mg of polylactic-co-glycolic acid (PLGA) was dissolved in 3 mL of chloroform.

Next, a solution of chitosan (0.25%, w/v) and PVA (2%, w/v) in cold distilled H₂O was mixed with 25 μl chloroform. This mixture was centrifuged at 1000 rpm for 5 minutes, and filtered using a 0.2 μm syringe filter such as a hydrophilic polysulfonic membrane syringe filter (25 mm Nalgene® filter unit, Nalge Co, Rochester, NY). This removes undissolved PVA. This step is important to increase microRNA loading efficiency, reduce nanoparticle aggregation, have better uniform nanoparticles (improved particle dispersion index) and regulate the particle size.

Next, 300 μl miR-210 H₂O solution (10% w/v) was added to the PLGA solution preferred as described above and the mixture was vortexed for 1 minute.

The miRNA solution was then placed in an ice bath for five minutes and then sonicated at 55W for 60 seconds. This step does not destroy the particles.

The miR-210 solution prepared as described above was then added in two portions to 12 mL of the PVA solution prepared as described above with vortexing. The resulting mixture was sonicated for two minutes. Then the mixture was stirred overnight, using a magnetic stirrer, to evaporate chloroform.

Following the overnight stir, the PNP suspension was stirred in a vacuum dedicator for one hour. The suspension was then transferred and centrifuged, preferably at 20,000 rpm for twenty minutes at 4° Celsius. The centrifuge is used to ensure the removal of leftover solvents. The pellet was then resuspended in distilled H₂O and sonicated for preferably sixty seconds in an ice bath. The wattage can be variable but 55W was used for optimal conditions. These steps-following the overnight stir-were repeated two additional times to remove PVA and unencapsulated miR-210.

After the last centrifuge step, the pellet was resuspended in 7 mL distilled H₂O, then sonicated for sixty seconds on an ice bath. The resuspension was then centrifuged at 1000 rpm for 10 minutes at 4° C. to remove large aggregates. The supernatant was collected and freeze-dried using glucose (2% w/v) as cryoprotectant, and subsequently lyophilized for two days. A freeze dryer (VirTis Co, Inc, Gardiner, NY) was used. Glucose has the best results in reducing nanoparticle aggregation and also improves lyophilization for injection. The lyophilized particles were stored at 4° C. with desiccant.

### Example 2

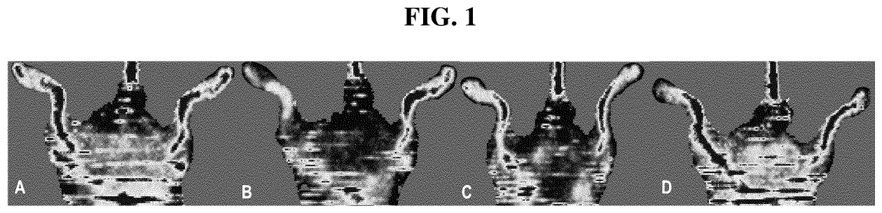

An example of the polymeric nanoparticle delivery system as described above was prepared, using the preferred components and miR-210 mimetic. C57BL/6J 18-month-old mice (n=6 per group) were fed with a high fat diet for 12 weeks, then underwent double ligation of the left femoral artery (HLI). The mice were injected intramuscularly with the polymeric nanoparticles, carrying miR-210, in a 5 μmol concentration per ˜0.160 grams of tissue, which is about the weight of the gastrocnemius. Mice were sacrificed at baseline, 2 weeks and 4 weeks post injection after taking pictures with a doppler blood flow imager. FIG. 1 shows images of blood flow Doppler imaging at rest, (A) before the HLI, (B) immediately after the HLI, (C) two weeks after HLI and (D) four weeks after HLI, where more shading means more blood flow.

As seen in FIG. 1, blood flow was increased 2 weeks post hind limb ligation and seemed to be completely recovered 4 weeks post hind limb ligation. The adductors were extracted from the ligated limb and mitochondrial respiration and reactive oxygen species (H₂O₂) production were measured using an Oroboros O2k Oxygraph FluoRespirometer (Oroboros Instruments, Innsbruck, Austria) consisting of two temperature-controlled chambers, each containing a polarographic oxygen sensor and a custom-fitted fluorometer (O2k-Fluo LED2 module). Mitochondrial oxygen consumption (JO2) and H2O2 emission (JH2O2) were simultaneously measured in saponin-permeabilized adductor muscle fibers. Experiments were performed at 37° C. in 2 mL of the respiration buffer (7.23 mM dipotassium egtazic acid (K2-EGTA), 2.77 mM Ca-EGTA, 20 mM imidazole, 20 mM taurine, 5.7 mM adenosine triphosphate, 14.3 mM phosphocreatine, 50 mM MES potassium salt, and 6.56 mM magnesium chloride hexahydrate, pH 7.1) with creatine monohydrate (20 mM). Fiber bundles were weighed immediately prior to their placement in each Oroboros chamber (approximately 10 mg wet weight per chamber) and ran in duplicate. A substrate inhibitor titration protocol was performed, whereby 2 mM malate and 10 mM glutamate were added to the chambers to measure Complex I, state 2 respiration. This was followed by the addition of 4 mM ADP, to initiate state 3 (ADPstimulated) respiration. Next, 10 mM succinate was added to the chambers to stimulate electron flow through Complex II. Rotenone (10 μM) was then used to inhibit Complex I, and 1 mM of duroquinol was added to measure Complex III. Finally, we added 5 μM antimycin A to inhibit Complex III. followed by 0.4 mM N,N,N′,N′-tetramethyl-p-phenylenediamine (TMPD), and 2 mM ascorbate to prevent TMPD auto-oxidation to measure Complex IV. All reagents and chemicals were purchased from Sigma Aldrich (St. Louis, MO).

The net rate of mitochondrial JH2O2 release per electron transport chain (ETC) complex was measured simultaneously during respirometry experiments using Amplex UltraRed reagent (AmR; 5 μM) (Thermo Scientific, Rockford, IL, A36006) and horseradish peroxidase (HRP; 1 U/mL) as described in previous publications. Briefly, after calibration with H2O2, JH2O2 was detected fluorometrically by monitoring the accumulation of resorufin at each stage of the substrate inhibitor titration protocol. Resorufin is the stable fluorescent product of 1:1 oxidation of AmR by H2O2 in the presence of HRP (at within-chamber excitation/emission wavelengths of 563/587 nm). Mitochondrial JO2 and JH2O2 are expressed as picomoles per second, normalized to wet-weight (pmols/see/wet weight).

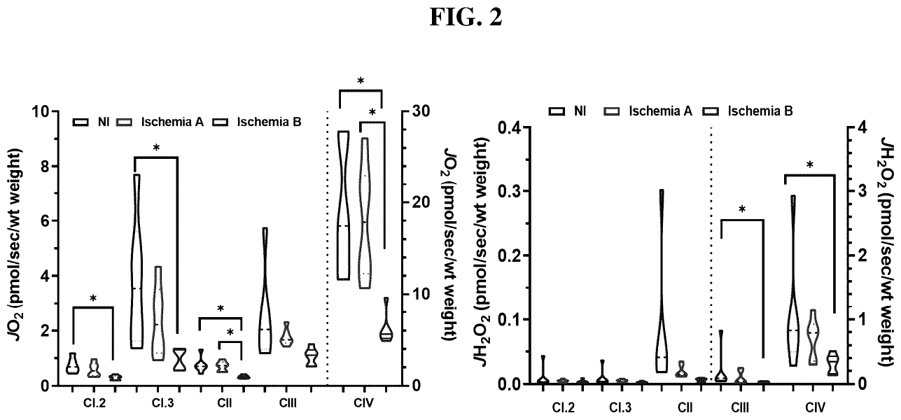

FIG. 2 shows the net rate of mitochondrial oxygen consumption (JO2) (left panel) and H₂O₂ emission (JH2O2) (right panel) of the non-ischemic limb (NI as the absolute control; left), ischemic limb at 2 weeks post injection (Ischemia A; middle) and 4 weeks post injection (Ischemia B; right). For Complexes state 2 (CI.2; basal) and state 3 (CI.3; maximum) the net rate of mitochondrial respiration was significantly different when comparing the NI with the Ischemia B time point. For Complexes II and IV the net rate of mitochondrial respiration was significantly reduced in Ischemia B compared with NI and Ischemia A. The H₂O₂ mitochondrial emission net rate was reduced after injecting with miR-210. However, there was a significant reduction in Ischemia B for both Complexes III and IV when comparing with the NI leg. These are important findings because under the same model with no miR-210 injections, mitochondrial respiration is reduced but H₂O₂ emission net rate is increased. Therefore, miR-210 has the ability to regulate mitochondrial oxidative stress under ischemic conditions.

### Example 3

An example of the polymeric nanoparticle delivery system as described above was prepared, using the preferred components and miR-210 mimetic and inhibitor. C57BL/6J 18-month-old mice (n=3 per group) were fed with a high fat diet for 12 weeks, then underwent double ligation of the left femoral artery (HLI). The mice were injected intramuscularly with the polymeric nanoparticles, carrying either miR-210 mimetic or inhibitor, in a 5 μmol concentration per ˜0.160 grams of tissue, which is about the weight of the gastrocnemius. Mice were sacrificed at baseline, post ligation, three days, seven days, fourteen days, twenty-one days and twenty-eight days post injection after taking pictures with a doppler blood flow imager. The RNeasy Mini Kit (74104, Qiagen, Hilden, Germany) and miRNeasy Mini Kit (Qiagen, 217084) were used to purifytotal RNA and miR from gastrocnemius tissue homogenates. A Taqman Advanced miRNA cDNA Synthesis Kit (Applied Biosystems, Waltham, MA, A28007) was used to prepare cDNA templates for miR assays. MiR-210 gene expression was analyzed by real-time PCR using a Taqman Advanced miRNA Assay kit (Applied Biosystems, A25576) run on a CFX Opus PCR system (Bio-Rad). The following miR-210 primer was used: mimetic mmu-miR-210-3p, mature miR sequence 5′-CUGUGCGUGUGACAGCGGCUGA-3′ (SEQ ID NO:1) and inhibitor mmu-miR-210-5p 5′-AGCCACUGCCCACCGCACACUG-3′ (SEQ ID NO: 2). Threshold cycle (CT) values between miR-210 and the endogenous control U6B small nuclear RNA (RNU6B) (Thermo Fisher Scientific, 001093) gene were used to normalize data. The 2\hat{ }(-delta delta CT) method was used to calculate fold change.

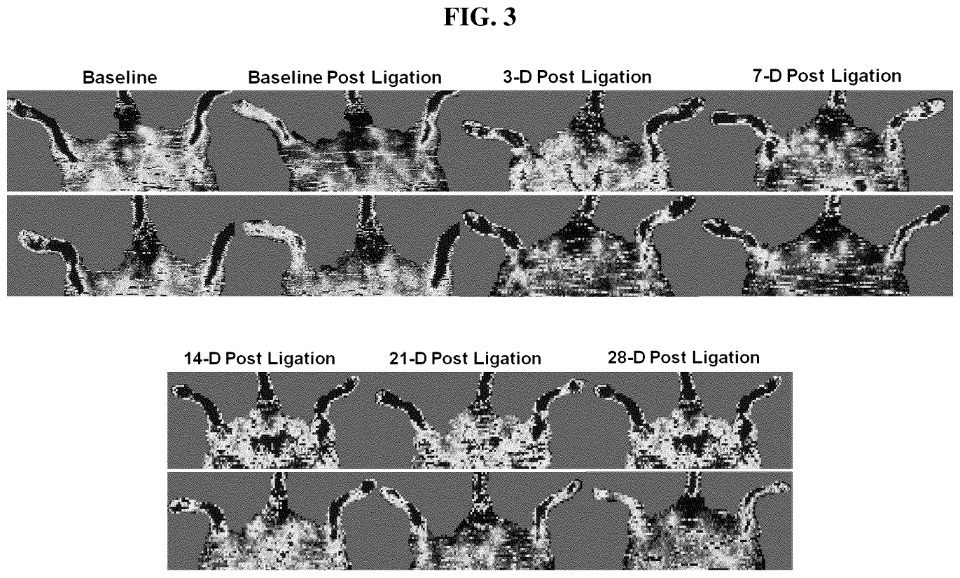

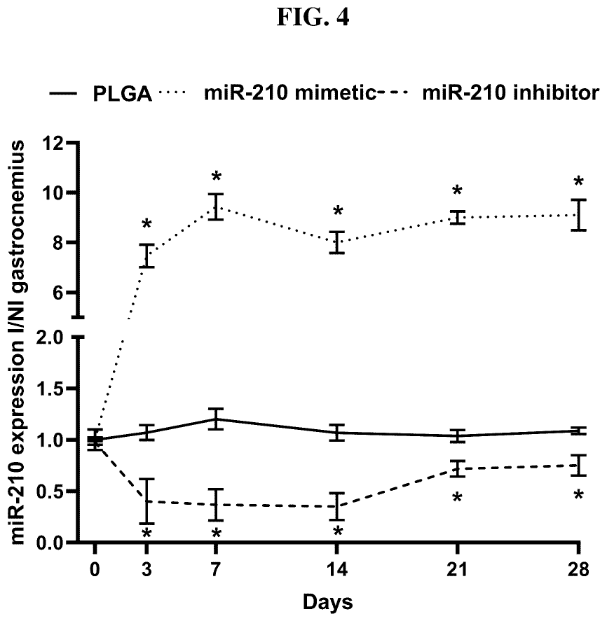

FIG. 3 shows images of blood flow Doppler imaging at the different time points, miR-210 mimetic top row and miR-210 inhibitor bottom row. It is evident that miR-210 mimetic increases blood flow significantly after 7-D till 28-D compared with the inhibitor that shows no difference in blood flow. There is significant improvement of blood flow when treating the mice with miR-210 mimetic after the seventh day. Mir-210 inhibitor demonstrates significantly reduced blood flow. FIG. 4 demonstrates that a one-time injection of the polymeric nanoparticle delivery system of miR-210 mimetic increases miR-210 expression significantly for at least 28 days post injection. Similarly, miR-210 inhibitor decreases significantly and PLGA alone (no miR) remains unchanged. It is evident that one time injection increases miR-210 gene expression in the gastrocnemius for at least 28 days post injection.

What is claimed is:

1. A method for treating cardiovascular disease in a subject, comprising:
administering to the subject a therapeutically effective amount of a drug delivery composition comprising polymeric nanoparticles and miR-210, wherein the polymeric nanoparticles comprise carboxylic acid polymers and chitosan, and wherein the polymeric nanoparticles encapsulate miR-210.

2. The method of claim 1, wherein the carboxylic acid polymers comprise polylactic-co-glycolic acid.

3. The method of claim 1, wherein the cardiovascular disease is peripheral artery disease (PAD).

4. The method of claim 1, wherein the miR-210 is miR-210 mimetic or miR-210 inhibitor.

5. A method for preparing polymeric nanoparticles encapsulating miR-210, comprising:
dissolving carboxylic acid polymers in chloroform to form a first polymer solution;
mixing chitosan, polyvinyl alcohol, and chloroform to form a chitosan solution;
adding miR-210 to the first polymer solution to form a microRNA solution;
adding the microRNA solution to the chitosan solution to form a microRNA suspension;
stirring and centrifuging the microRNA suspension; and
collecting a supernatant following centrifuging of the microRNA suspension, wherein the supernatant comprises polymeric nanoparticles encapsulating miR-210.

6. The method of claim 4, wherein the carboxylic acid polymers comprise polylactic-co-glycolic acid.

7. The method of claim 4, further comprising freeze-drying and lyophilizing the supernatant.

8. The method of claim 4, wherein the step of adding the microRNA solution to the chitosan solution to form a microRNA suspension further comprises vortexing and sonicating the microRNA suspension and evaporating chloroform from the microRNA suspension.

9. The method of claim 4, wherein the step of stirring and centrifuging the microRNA suspension comprises a first centrifuging step to form a pellet and remove solvent, resuspending the pellet to form an intermediate suspension, sonicating the intermediate suspension, and a second centrifuging step to separate the intermediate suspension.

10. The nanoparticles encapsulating miR-210 prepared by the method of claim 4.

11. A pharmaceutical composition comprising the nanoparticles encapsulating miR-210 prepared by the method of claim 10.

12. A method for treating cardiovascular disease comprising administering a therapeutically effective amount of the pharmaceutical composition of claim 11 to a patient having cardiovascular disease.

13. The method of claim 12, wherein the cardiovascular disease is peripheral artery disease (PAD).


SEQUENCE LISTING (2 sequences)

SEQ ID NO: 1 - RNA, 22 nt, synthetic construct
  source 1..22: mol_type = other RNA
  modified_base 2: mod_base = OTHER; note = uracil
  modified_base 4: mod_base = OTHER; note = uracil
  modified_base 8: mod_base = OTHER; note = uracil
  modified_base 10: mod_base = OTHER; note = uracil
  modified_base 20: mod_base = OTHER; note = uracil
ctgtgcgtgtgacagcggctga

SEQ ID NO: 2 - RNA, 22 nt, synthetic construct
  source 1..22: mol_type = other RNA
  modified_base 7: mod_base = OTHER; note = uracil
  modified_base 21: mod_base = OTHER; note = uracil
agccactgcccaccgcacactg